(19) United States
(12) Patent Application Publication
(10) Pub. No.: US 2022/0145351 A1
(43) Pub. Date: May 12, 2022

(54) METHOD FOR DETECTING INFECTIVITY OF HUMAN CORONAVIRUS

(71) Applicant: Delta Electronics Int'l (Singapore) Pte Ltd, Singapore (SG)

(72) Inventors: Yong Zhang, Singapore (SG); Jia Hui Esther Boey, Singapore (SG); Weishi Zhang, Singapore (SG); Quanke Long, Singapore (SG); Jiaojiao Li, Singapore (SG); Xiaozhi Zhao, Singapore (SG); Kun-Nan Tsai, Singapore (SG)

(21) Appl. No.: 17/520,070

(22) Filed: Nov. 5, 2021

(30) Foreign Application Priority Data
Nov. 12, 2020 (SG) 10202011273P
Oct. 26, 2021 (CN) 202111248349.3

(51) Int. Cl. C12Q 1/06

(52) U.S. Cl. CPC C12Q 1/06; C12Q 2531/113; G01N 2800/26

(57) ABSTRACT

A method for detecting infectivity of a human coronavirus is provided. The method includes steps of: (a) dividing a testing sample into a first sample and a second sample; (b) treating the first sample with an intercalating dye or chemical; (c) exposing the first sample to a light for photo-activation; (d) amplifying targeted nucleic acids in the first sample and the second sample; and (e) determining infectivity of the human coronavirus based on amplification results of the first sample and the second sample.

### FIELD OF THE INVENTION

The present invention relates to a diagnosis of a human coronavirus, and more particularly to a method for detecting infectivity of a human coronavirus.

### BACKGROUND OF THE INVENTION

Traditional culture and microscopy methods for detection of viable cells can be tedious, labor-intensive and time-consuming. Some methods enable viability to be assessed by staining techniques, such as BacLight fluorescence microscopy or acridine orange, flow cytometry coupled with dyes, and physiological tests for cellular respiration, which are not capable of detection of specific pathogen species. These culture-based methods give rise to several challenges such as the isolation and identification of specific pathogens among a plethora of background microflora, and the detection of pathogens that occur at low levels, the potential biosafety issues to the operators and environment that may be introduced during tedious manual processes. In addition, the culture-based methods encounter another issue that some human pathogens may enter a “viable but non-culturable” (VBNC) physiological state, in which they are living but cannot be grown outside of their natural habitat.

Molecular methods targeting nucleic acids have revolutionized microbial detection. DNA/RNA-based methods, such as polymerase chain reaction (PCR) or reverse transcriptase polymerase chain reaction (RT-PCR), are rapid, versatile, sensitive, precise, and allow specific detection and/or quantification of microorganisms of interest in food, environmental, and clinical samples. Despite these advantages, broad application is still hampered by some challenges. The inability to differentiate between viable and nonviable cells and the resulting in overestimation of microbial targets is considered a major disadvantage of nucleic acid based detection methods.

The novel coronavirus called severe acute respiratory syndrome coronavirus 2 (SARS-CoV-2), emerging from December 2019, was detected through real-time RT-PCR (qRT-PCR) with primers against segments of its RNA genome. qRT-PCR has demonstrated high sensitivity, specificity, and ability to deliver reliable quantitative/qualitative data in clinical samples, although such results do not indicate the infectivity of detected viruses. Many laboratories choose to assay the presence of viral genomes by PCR/RT-PCR. This is an acceptable technique as long as the limitations are understood—it detects nucleic acids, not infectious virus.

One of the criteria that should be met for Covid-19 patients for hospital discharge or discontinuation of quarantines: At least 2 consecutively negative results of RT-PCR testing separated by at least a 24-hour interval. However, in some cases, the Covid-19 patients could not be discharged from hospitals because of persistence of positive RT-PCR results, though all other clinical symptoms disappeared already. Experts have also raised doubts about post-recovery testing using the current RT-PCR method. There is concern that such tests are being misinterpreted to suggest people are infectious when they probably are not, and are keeping them from returning to work. Multiple studies have now shown that some people who have recovered from the illness will nonetheless test positive for long periods by PCR/RT-PCR, which looks for fragments of the virus' DNA/RNA in mucus swabbed from deep in nasal passages.

There is an urgent need for a method which is able to quickly and precisely discriminate viable and inviable SARS-CoV-2. This will be very important supporting information for doctors in managing Covid-19 patients. Cell culture systems and animal models have not proven to be reliable or practical yet.

### SUMMARY OF THE INVENTION

An object of the present invention is to provide a method for detecting SARS-CoV-2 infectivity by viability PCR to quickly and precisely discriminate viable and inviable SARS-CoV-2.

Another object of the present invention is to provide a method for detecting infectivity of a human coronavirus by viability PCR to quickly and precisely discriminate viable and inviable virus.

An additional object of the present invention is to provide a method for evaluating membrane or envelope integrity of a human coronavirus so as to determine infectivity of the human coronavirus.

In accordance with an aspect of the present invention, a method for detecting infectivity of a human coronavirus is provided. The method includes steps of: (a) dividing a testing sample into a first sample and a second sample; (b) treating the first sample with an intercalating dye or chemical; (c) exposing the first sample to a light for photo-activation; (d) amplifying targeted nucleic acids in the first sample and the second sample; and (e) determining infectivity of the human coronavirus based on amplification results of the first sample and the second sample.

In accordance with an aspect of the present invention, a method for detecting evaluating membrane or envelope integrity of a human coronavirus is provided. The method includes steps of: (a) dividing a testing sample into a first sample and a second sample; (b) treating the first sample with an intercalating dye or chemical; (c) exposing the first sample to a light for photo-activation; (d) amplifying targeted nucleic acids in the first sample and the second sample; and (e) determining membrane or envelope integrity of the human coronavirus based on amplification results of the first sample and the second sample.

The above contents of the present invention will become more readily apparent to those ordinarily skilled in the art after reviewing the following detailed description and accompanying drawings, in which:

### BRIEF DESCRIPTION OF THE DRAWINGS

FIGS. 1 and 2 show schematic work flows of viability qPCR/qRT-PCR for virus infectivity detection;

FIGS. 3 and 4 show experimental data for detecting HCoV-229E infectivity by viability qRT-PCR;

FIG. 5 shows experimental data for detecting HCoV-OC43 infectivity by viability qRT-PCR; and

FIG. 6 shows experimental data for detecting SARS-CoV-2 infectivity by viability qRT-PCR.

### DETAILED DESCRIPTION OF THE PREFERRED EMBODIMENT

The present invention will now be described more specifically with reference to the following embodiments. It is to be noted that the following descriptions of the embodiments of this invention are presented herein for purpose of illustration and description only; it is not intended to be exhaustive or to be limited to the precise form disclosed.

The present invention is to develop a method in order to discriminating infectious and inactivated/dead virus in clinical samples, thereby provide clinical diagnostic information for Covid-19 patient management and therapy. This method uses a nucleic acid intercalating dye or chemical that selectively enters cells with compromised cell membranes, whereas an intact cell membrane presents a barrier for this molecule. Once inside a (dead) cell, the dye or chemical intercalates into the cell's nucleic acid to which it is believed to covalently crosslink after exposure to strong visible light due to the presence of an azide group. Photolysis converts the azide group into a highly reactive nitrene radical, which can react with any organic molecule in its proximity. Reaction with nucleic acid can be assumed to occur with a high probability considering the spatial proximity of the intercalating dye or chemical. The modification was empirically found to strongly inhibit its amplification. At the same time when the cross-linking with nucleic acid occurs, any unbound excess dye or chemical reacts with water molecules. The resulting hydroxylamine is no longer reactive, preventing reaction of the dye or chemical with nucleic acid extracted from intact cells. Therefore, by this mechanism, the dye or chemical can preferably intercalate nucleic acid of the dead cells and thus prevent subsequent nucleic acid amplification of dead cells by PCR/RT-PCR.

PCR/RT-PCR procedure, the gold standard for infectious pathogen detection, combined with intercalating dye or chemical treatment, generates a promising technique (Viability PCR/RT-PCR) which makes use of the speed and sensitivity of the molecular detection, while at the same time providing viability information. The method (Viability PCR/RT-PCR) is well applied to detect the infectivity of bacteria and viruses (norovirus, hepatitis A virus, bacteriophage) in food and environmental samples, but has not been reported in the aspects of detecting infectivity of SARS-CoV-2 or other human coronavirus.

Therefore, the present invention is to develop optimized viability PCR/RT-PCR method, preferably qPCR/qRT-PCR for real-time detection, which is applied to clinical samples to assess its performance in discriminating between potentially infectious and inactivated viral particles, thereby provide more supporting information for SARS-CoV-2 or other human coronavirus diagnosis and therapy.

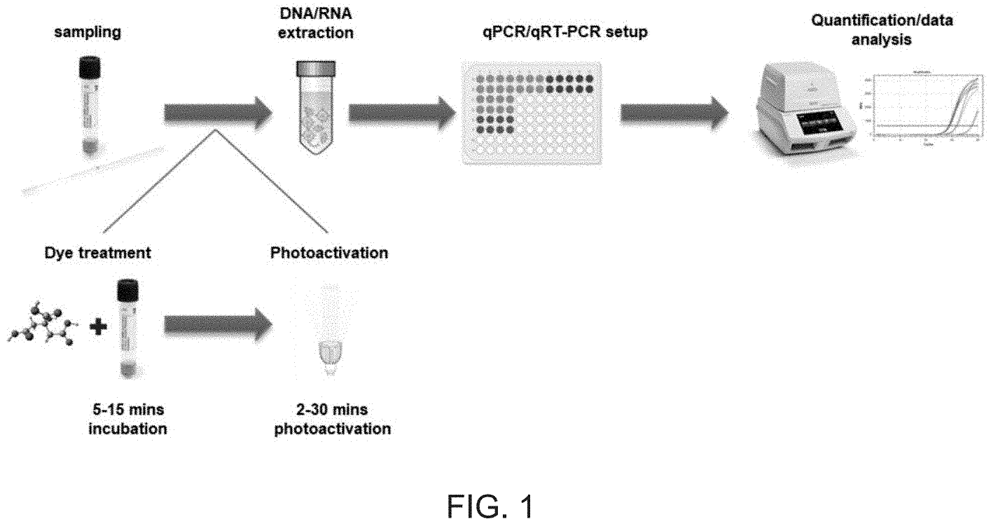

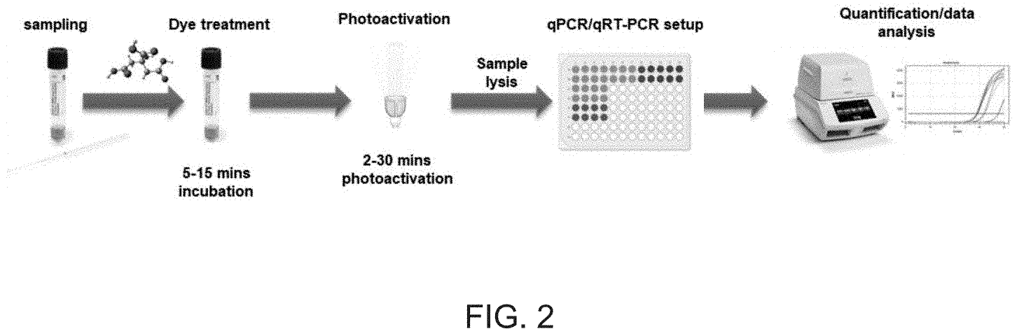

Accordingly, the present invention provides a method for rapidly detecting infectivity of a human coronavirus, e.g. HCoV-229E, HCoV-NL63, HCoV-OC43, HCoV-HKU1, MERS-CoV, SARS-CoV, and SARS-CoV-2. FIGS. 1 and 2 show schematic work flows of viability qPCR/qRT-PCR for virus infectivity detection. As shown in FIGS. 1 and 2, the steps of dye or chemical treatment and photoactivation are added before DNA/RNA extraction in standard qPCR/qRT-PCR based diagnosis or directly added into amplification reaction for direct qPCR/qRT-PCR based diagnosis. Since the coronavirus is RNA virus, the method of the present invention uses viability qRT-PCR for detecting coronavirus infectivity.

On the other hand, the nucleic acid intercalating dye or chemical only enters the cells with compromised membranes. Therefore, the present invention also provides a method for evaluating membrane or envelope integrity of a human coronavirus, so as to determine infectivity of the human coronavirus. The method is described in detail as follows.

Before performing the viability qRT-PCR, the following reagents or systems should be prepared: (a) an intercalating dye or chemical, which can covalently crosslink with nucleic acid when exposure to a visible light; (b) a photo-activation system; (c) a buffer system which can extract the nucleic acid from the sample or a buffer system which can directly extract and amplify the targeted nucleic acid; (d) a set of primers with or without one or more probes targeted to the virus; (e) nucleic acid amplification system; and (e) temperature controlling system.

The method includes the following steps. First, a testing sample is divided into a first sample and a second sample. Subsequently, an intercalating dye or chemical is added into the first sample followed by incubation in the dark at room temperature. Then, the first sample is exposed to a light for photo-activation. Afterward, the above-mentioned buffer system is mixed with both the first sample and the second sample separately. Then, both the first sample and the second sample are performed with nucleic acid amplification with the above-mentioned primers and probes and the nucleic acid amplification system under optimized thermal cycling profile. Finally, the infectivity or the membrane/envelope integrity of the human coronavirus is determined based on nucleic acid amplification results from the first sample and the second sample.

In an embodiment, the second sample is not subject to dye treatment and photo-activation. Alternatively, the second sample is subject to dye treatment but not subject to photo-activation. Or, the second sample is not subject to dye treatment but subject to photo-activation.

The criteria for the presence of the non-infectious virus in samples includes: (1) the Cq value of the sample treated by the intercalating dye or chemical is larger than that of the sample not treated by the dye or chemical or the sample treated by the dye or chemical but not photo-activated, and the sample contains inactivated virus; (2) the larger the ΔCq, the higher the inactivated virus content; (3) the ΔCq is at reasonably defined range (eg. more than or equal to 5-8) or no typical amplification curve from the sample treated by the intercalating dye or chemical, or the Cq is more than the cut-off value of the assay, which indicates that the virus in the sample has been completely inactivated.

In an embodiment, the intercalating dye or chemical is PMA (propidium monoazide) dye, PMAxx dye, or dye with similar functions, such as EMA (ethidium monoazide), platinum compounds, palladium compounds, or Reagent D.

In an embodiment, the first sample is exposed to a strong visible light for photo-activation.

In an embodiment, the wavelength of the light is from 450 to 480 nm, and the light intensity is from 85 to 250 mW.

In an embodiment, the dye treatment duration is from 5 to 15 min.

In an embodiment, the photo-activation duration is from 2 to 30 min.

In an embodiment, the temperature control during photo-activation is less than 37° C.

In an embodiment, the sample can be extracted with the buffer system followed by nucleic acid purification and amplification.

In an embodiment, the sample can be directly mixed with the buffer system that combines both nucleic acid extraction and amplification steps.

In an embodiment, the nucleic acid amplification method can be PCR/RT-PCR, or isothermal based amplification methods.

In an embodiment, the nucleic acid amplification method can be real-time or end point detection.

In an embodiment, the calculation formula to determine the infectivity or the membrane/envelope integrity of the human coronavirus is based on Cq difference (ΔCq) between the first sample (dye treated and photo-activated sample) and the second sample.

In an embodiment, when the targeted nucleic acid is DNA, it is amplified with a pair or multiple pairs of primers for the targeted DNA.

In an embodiment, when the targeted nucleic acid is RNA, it is reverse transcribed and amplified with a pair or multiple pairs of primers for the targeted RNA. In this situation, a reverse transcriptase is provided in the system for RNA to be reverse transcribed to cDNA.

In an embodiment, the targeted nucleic acid is detected by one or more probes specific to the targeted sequence.

In an embodiment, the human coronavirus is HCoV-229E, HCoV-NL63, HCoV-OC43, HCoV-HKU1, MERS-CoV, SARS-CoV, or SARS-CoV-2.

In an embodiment, the primer pair targeting SARS-CoV-2 includes a forward primer 5′-ACAGGTACGTTAATAGTTAATAGCGT-3′ (SEQ ID NO: 1) and a reverse primer 5′-ATATTGCAGCAGTACGCACACA-3′ (SEQ ID NO: 2).

In an embodiment, the primer pair targeting SARS-CoV-2 includes a forward primer 5′-CACATTGGCACCCGCAATC-3′ (SEQ ID NO: 3) and a reverse primer 5′-GAGGAACGAGAAGAGGCTTG-3′ (SEQ ID NO: 4).

In an embodiment, the primer pair targeting HCoV-229E includes a forward primer 5′-GCTTTACGTTGACGGACATAGA-3′ (SEQ ID NO: 5) and a reverse primer 5′-CGGACCTTCCGACTCTACTATAA-3′ (SEQ ID NO: 6).

In an embodiment, the primer pair targeting HCoV-OC43 includes a forward primer 5′-GAACTATGGCATTTGGATACAGG-3′ (SEQ ID NO: 7) and a reverse primer 5′-ATGACTGCAAATAGCCCAAATT-3′ (SEQ ID NO: 8).

The following is an example illustrating the method for detecting SARS-CoV-2 infectivity. First, a clinical sample was mixed thoroughly with an intercalating dye PMA/PMAxx (final concentration is 50-1000 μM) in DNA LoBind 1.5 ml tubes and incubated in the dark at room temperature for 5-15 min. Another sample was mixed with water or PBS buffer and used as a control.

Then, the samples were immediately exposed to photo-activation using a photo-activation system for 2-30 minutes to ensure that the PMA/PMAxx and the nucleic acid are fully covalently crosslinked.

After viability dye treatment and photo-activation, viral RNA was extracted and amplified by qRT-PCR specific to SARS-CoV-2. qRT-PCR reaction setup includes: reverse transcriptase mix, PCR master mix, forward primer, reverse primer, probe, template RNA, top up to with PCR-grade water. qRT-PCR protocol was conducted based on requirement. For example, the forward primer targeting E gene is 5′-ACAGGTACGTTAATAGTTAATAGCGT-3′ (SEQ ID NO: 1) and the reverse primer targeting E gene is 5′-ATATTGCAGCAGTACGCACACA-3′ (SEQ ID NO: 2). Alternatively, the forward primer targeting N gene is 5′-CACATTGGCACCCGCAATC-3′ (SEQ ID NO: 3) and the reverse primer targeting N gene is 5′-GAGGAACGAGAAGAGGCTTG-3′ (SEQ ID NO: 4).

The following is another example illustrating the method for detecting HCoV-229E infectivity. The steps are similar to those for detecting SARS-CoV-2 infectivity, but the primers and probes used in qRT-PCR are specific to HCoV-229E. For example, the forward primer targeting HCoV-229E is 5′-GCTTTACGTTGACGGACATAGA-3′ (SEQ ID NO: 5) and the reverse primer targeting HCoV-229E is 5′-CGGACCTTCCGACTCTACTATAA-3′ (SEQ ID NO: 6).

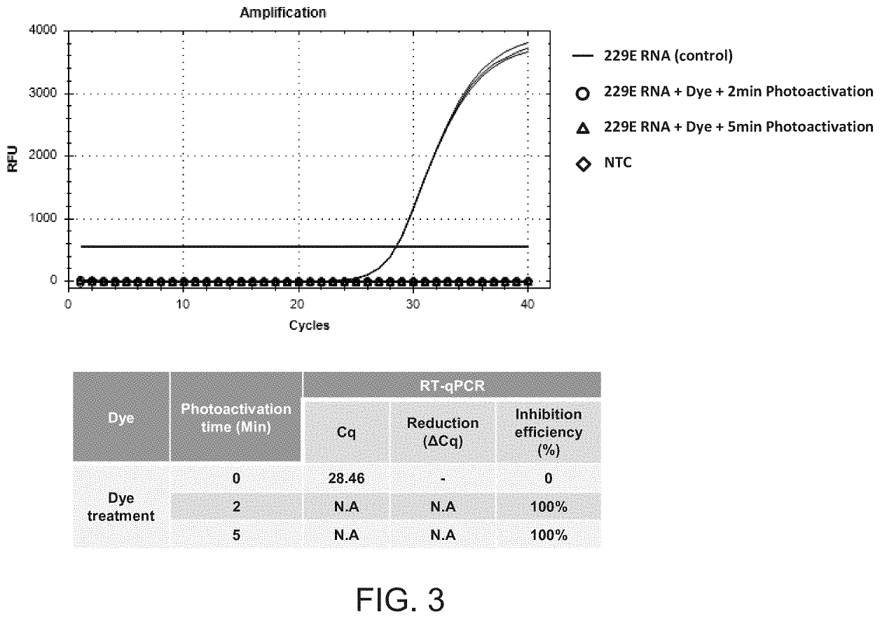

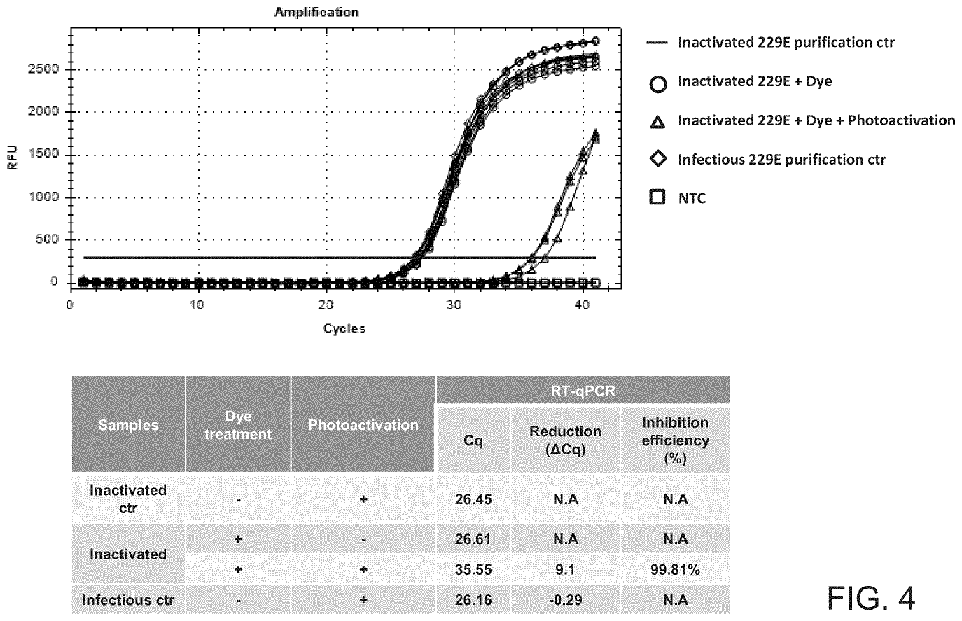

FIGS. 3 and 4 show experimental data for detecting HCoV-229E infectivity by viability qRT-PCR, wherein the experiment of FIG. 3 used viral RNA as target, and the experiment of FIG. 4 used HCov-229E as target. As shown in FIG. 3, since viral RNA is purified nucleic acid without enveloped membrane, when the viral RNA was dye treated and photo-activated, no amplification was resulted. As shown in FIG. 4, when the viable (infectious) virus was inactivated by heat, for example by heating to 65-75° C. for 30 minutes, most cells became dead, which resulted in delayed amplification (ΔCq was 9.1) after dye treated and photo-activated. While in the inactivated 229E purification control, the infectious 229E purification control, and the inactivated 229E with dye treated but no photo-activated, normal amplifications were resulted. Therefore, the method of the present invention is able to differentiate viable and dead cells for HCoV-229E by viability qRT-PCR, so as to detect the infectivity of the human coronavirus.

From FIGS. 3 and 4, it is also proved that RNA amplification can be inhibited by dye treatment and photo-activation during viability qRT-PCR. Although RNA is single-stranded nucleic acids, it has some double-stranded structures, e.g. hairpin or stem-loop resulted from intramolecular base pairing, and thus may be bonded by the intercalating dye.

The following is a further example illustrating the method for detecting HCoV-OC43 infectivity. Similar to SARS-CoV-2, HCoV-OC43 also belong to beta coronavirus. The steps are similar to those for detecting SARS-CoV-2 infectivity, but the primers and probes used in qRT-PCR are specific to HCoV-OC43. For example, the forward primer targeting HCoV-OC43 is 5′-GAACTATGGCATTTGGATACAGG-3′ (SEQ ID NO: 7), the reverse primer targeting HCoV-OC43 is 5′-ATGACTGCAAATAGCCCAAATT-3′ (SEQ ID NO: 8), and the probe targeting HCoV-OC43 is 5′-TGCTATACCCAATGGCAGGAA-3′ (SEQ ID NO: 9).

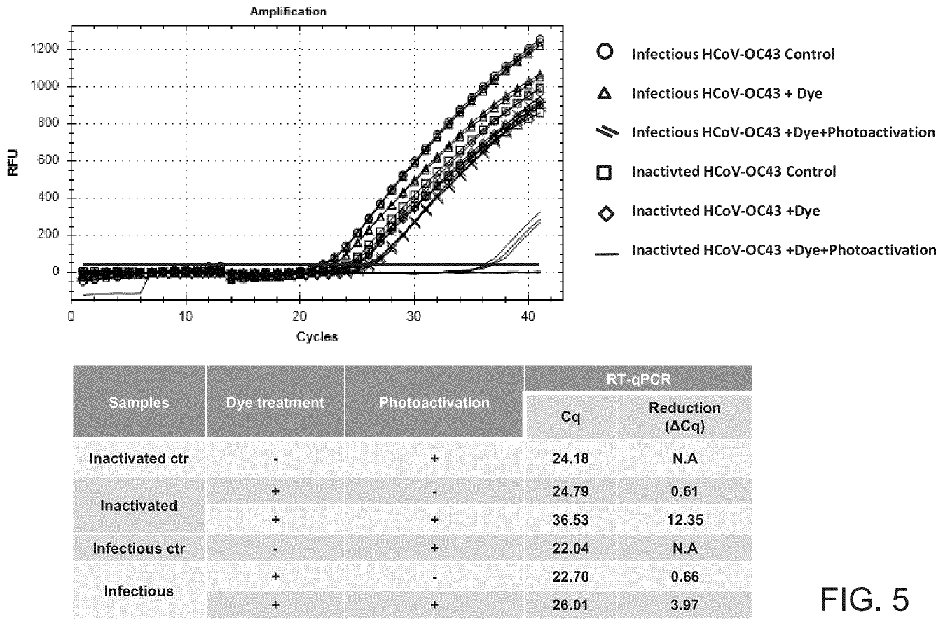

FIG. 5 shows experimental data for detecting HCoV-OC43 infectivity by viability qRT-PCR. As shown in FIG. 5, when the viable (infectious) virus was inactivated by heat, for example by heating to 65-75° C. for 30 minutes, most cells became dead, which resulted in delayed amplification (ΔCq was 12.35) after dye treated and photo-activated. While in the infectious HCoV-OC43 control, the infectious HCoV-OC43 with dye treated but no photo-activated, the infectious HCoV-OC43 with dye treated and photo-activated, the inactivated HCoV-OC43 control, and the inactivated HCoV-OC43 with dye treated but no photo-activated, normal amplifications were resulted. Among them, since the infectious HCoV-OC43 may include some naturally dead cells, small Cq reduction was resulted after dye treated and photo-activated. Therefore, the method of the present invention is able to differentiate viable and dead cells for HCoV-OC43 by viability qRT-PCR, so as to detect the infectivity of the human coronavirus.

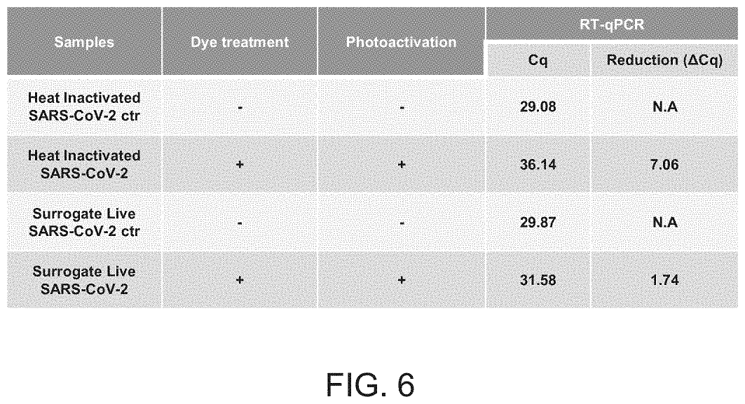

The following is an additional example illustrating the method for detecting SARS-CoV-2 infectivity. In this example, chemically modified viral particles were used as surrogate live viruses, which is formulated with purified, intact viral particles and chemically modified to render them non-infectious and refrigerator stable. FIG. 6 shows experimental data for detecting SARS-CoV-2 infectivity by viability qRT-PCR. As shown in FIG. 6, when the surrogate live SARS-CoV-2 was inactivated by heat, for example by heating to 65-75° C. for 30 minutes, most cells became dead, which resulted in delayed amplification (ΔCq was 7.06) after dye treated and photo-activated. While in the heat inactivated SARS-CoV-2 control, the surrogate live SARS-CoV-2 control, and the surrogate live SARS-CoV-2 with dye treated and photo-activated, normal amplifications were resulted. Among them, since the surrogate live SARS-CoV-2 may include some naturally dead cells, small Cq reduction was resulted after dye treated and photo-activated. Therefore, the method of the present invention is able to differentiate viable and dead cells for SARS-CoV-2 by viability qRT-PCR, so as to detect the infectivity of the human coronavirus.

Therefore, when compared with the prior art, the method for detecting human coronavirus infectivity of the present invention by viability qRT-PCR has the advantages of having a short detection time, high specificity, reliable results, simple data analysis, most importantly, differentiation of live and dead viruses. Compared with culture-based methods, the present invention increases the practicability of infectious SARS-CoV-2 detection, which is able to quickly and precisely discriminate viable and inviable SARS-CoV-2 with much faster turnaround time, lower biosafety risk, and can be used for detecting samples such as tissue samples, swab, serum, plasma, and thus it is suitable for prevention and control units at all levels of laboratories.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A method for detecting infectivity of a human coronavirus, comprising steps of:
(a) dividing a testing sample into a first sample and a second sample;
(b) treating the first sample with an intercalating dye or chemical;
(c) exposing the first sample to a light for photo-activation;
(d) amplifying targeted nucleic acids in the first sample and the second sample; and
(e) determining infectivity of the human coronavirus based on amplification results of the first sample and the second sample.

2. The method according to claim 1, wherein the second sample is not subject to dye treatment and photo-activation.

3. The method according to claim 1, wherein the second sample is subject to dye treatment but not subject to photo-activation.

4. The method according to claim 1, wherein the second sample is not subject to dye treatment but subject to photo-activation.

5. The method according to claim 1, wherein the intercalating dye or chemical is PMA dye, PMAxx dye, EMA, platinum compounds, palladium compounds, or Reagent D.

6. The method according to claim 1, wherein a duration of dye treatment in the step (b) is from 5 to 15 min.

7. The method according to claim 1, wherein a duration of photo-activation in the step (c) is from 2 to 30 min.

8. The method according to claim 1, wherein the first sample is exposed to a visible light for photo-activation in the step (c).

9. The method according to claim 1, wherein a wavelength of the light is from 450 to 480 nm.

10. The method according to claim 1, wherein an intensity of the light is from 85 to 250 mW.

11. The method according to claim 1, wherein a temperature control during photo-activation in the step (c) is less than 37° C.

12. The method according to claim 1, wherein the first sample and the second sample are extracted with a buffer system followed by nucleic acid purification and amplification.

13. The method according to claim 1, wherein the first sample and the second sample are directly mixed with a buffer system that combines both nucleic acid extraction and amplification steps.

14. The method according to claim 1, wherein the nucleic acids are amplified by PCR, RT-PCR, or isothermal based amplification methods.

15. The method according to claim 1, wherein the amplified nucleic acids are detected by real-time detection or end point detection.

16. The method according to claim 1, wherein a calculation formula for infectivity determination in the step (e) is based on Cq difference (ΔCq) between the first sample and the second sample.

17. The method according to claim 1, wherein the targeted nucleic acid is RNA, and it is reverse transcribed and amplified with a pair or multiple pairs of primers for the targeted RNA.

18. The method according to claim 1, wherein the targeted nucleic acid is detected by one or more probes.

19. The method according to claim 1, wherein the human coronavirus is HCoV-229E, HCoV-NL63, HCoV-OC43, HCoV-HKU1, MERS-CoV, SARS-CoV, or SARS-CoV-2.

20. The method according to claim 1, wherein a primer pair targeting SARS-CoV2 includes a forward primer 5′-ACAGGTACGTTAATAGTTAATAGCGT-3′ (SEQ ID NO: 1) and a reverse primer 5′-ATATTGCAGCAGTACGCACACA-3′ (SEQ ID NO: 2).

21. The method according to claim 1, wherein a primer pair targeting SARS-CoV2 includes a forward primer 5′-CACATTGGCACCCGCAATC-3′ (SEQ ID NO: 3) and a reverse primer 5′-GAGGAACGAGAAGAGGCTTG-3′ (SEQ ID NO: 4).

22. The method according to claim 1, wherein a primer pair targeting HCoV-229E includes a forward primer 5′-GCTTTACGTTGACGGACATAGA-3′ (SEQ ID NO: 5) and a reverse primer 5′-CGGACCTTCCGACTCTACTATAA-3′ (SEQ ID NO: 6).

23. The method according to claim 1, wherein a primer pair targeting HCoV-OC43 includes a forward primer 5′-GAACTATGGCATTTGGATACAGG-3′ (SEQ ID NO: 7) and a reverse primer 5′-ATGACTGCAAATAGCCCAAATT-3′ (SEQ ID NO: 8).

24. A method for evaluating membrane or envelope integrity of a human coronavirus, comprising steps of:
(a) dividing a testing sample into a first sample and a second sample;
(b) treating the first sample with an intercalating dye or chemical;
(c) exposing the first sample to a light for photo-activation;
(d) amplifying targeted nucleic acids in the first sample and the second sample; and
(e) determining membrane or envelope integrity of the human coronavirus based on amplification results of the first sample and the second sample.


SEQUENCE LISTING (9 sequences)

SEQ ID NO: 1 - DNA, 26 bp, Artificial Sequence
  Synthetically generated primer
acaggtacgttaatagttaatagcgt

SEQ ID NO: 2 - DNA, 22 bp, Artificial Sequence
  Synthetically generated primer
atattgcagcagtacgcacaca

SEQ ID NO: 3 - DNA, 19 bp, Artificial Sequence
  Synthetically generated primer
cacattggcacccgcaatc

SEQ ID NO: 4 - DNA, 20 bp, Artificial Sequence
  Synthetically generated primer
gaggaacgagaagaggcttg

SEQ ID NO: 5 - DNA, 22 bp, Artificial Sequence
  Synthetically generated primer
gctttacgttgacggacataga

SEQ ID NO: 6 - DNA, 23 bp, Artificial Sequence
  Synthetically generated primer
cggaccttccgactctactataa

SEQ ID NO: 7 - DNA, 23 bp, Artificial Sequence
  Synthetically generated primer
gaactatggcatttggatacagg

SEQ ID NO: 8 - DNA, 22 bp, Artificial Sequence
  Synthetically generated primer
atgactgcaaatagcccaaatt

SEQ ID NO: 9 - DNA, 21 bp, Artificial Sequence
  Synthetically generated probe
tgctatacccaatggcaggaa